(19) United States
(12) Patent Application Publication
(10) Pub. No.: US 2011/0306831 A1
(43) Pub. Date: Dec. 15, 2011

(54) ENDOSCOPE WITH A BENDING PORTION

(75) Inventors: Ole Køhnke, Frederiksberg (DK); Louise Wagner Petersen, Copenhagen (DK); Steen Borrye, Hillerod (DK)

(73) Assignee: AMBU A/S, Ballerup (DK)

(21) Appl. No.: 13/133,887

(22) PCT Filed: Dec. 9, 2009
(86) PCT No.: PCT/EP09/66726

§ 371 Date: Aug. 25, 2011

(30) Foreign Application Priority Data
Dec. 10, 2008 (DK) PA200801757

(51) Int. Cl. A61B 1/00; A61B 1/04

(52) U.S. Cl. 600/109; 600/121

(57) ABSTRACT

An endoscope (1) comprising a control handle (2), a flexible insertion portion (3) having length L₁ and being attached to the distal end of the control handle, a bending portion (4) having length L₂ and being attached to the distal end of the flexible insertion portion, a rigid tip portion (5) having length L₃ and being attached to the distal end of the bending portion, a control mechanism arranged to control the bending of the bending portion, and an elastic covering member (12) which is arranged to cover the bending portion and where the distal end of said elastic covering member is attached to the distal end (16) of said bending section and/or to said rigid tip portion (14) and where the proximal end of said elastic covering member is attached to the flexible insertion portion (18) and/or to the proximal end of the bending portion. The elastic covering member is arranged such that it is stretched when the bending portion is in its neutral position. In this way a low cost and flexible bending portion is provided.

The current invention relates to an endoscope having a distal end arranged to be inserted into a body cavity of a patient and a proximal end which is held by the user of the endoscope, said endoscope comprising a control handle at the proximal end, a flexible insertion portion having length L₁ and being attached to the distal end of the control handle, a bending portion having length L₂ and being attached to the distal end of the flexible insertion portion, a rigid tip portion having length L₃ and being attached to the distal end of the bending portion, a control mechanism arranged to control the bending of the bending portion, and an elastic covering member which is arranged to cover the bending portion and where the distal end of said elastic covering member is attached to the distal end of said bending section and/or to said rigid tip portion and where the proximal end of said elastic covering member is attached to the flexible insertion portion and/or to the proximal end of the bending portion. In particular the current invention deals with improvements to the bendable portion of an endoscope as mentioned above.

### DESCRIPTION OF RELATED ART

Endoscopes as described in the opening paragraph have been known for many years. A typical example of such an endoscope is described in EP 0 524 755. In this type of endoscope the bending section is comprised of a number of articulated links. The links are made from a metal plate element which is bent, pressed and punched into the correct shape. Successive links are pivotably joined together with rivets. This is a very strong construction but is expensive and complex to manufacture. In addition, the rivets take up space either inside the mechanism or outside the mechanism usually resulting in an increase in the outer diameter of the mechanism. Other forms of bending sections have also been presented. EP 1090581 for example shows a similar bending portion, but where holes and pins are formed in the links by pressing the plate material. The holes and pins can then be engaged to provide the pivotable connection between two successive links without the need for rivets.

Lately other forms of bending portions have been proposed with different materials such as plastic, see U.S. Pat. No. 6,743,239, U.S. Pat. No. 3,190,280 and EP 0626604 for example. In addition, bending portions have been proposed which are electronically conductive in order to shield the components arranged inside the bending portion, see for example U.S. Pat. No. 6,364,828.

Since bending portions are typically made from a plurality of articulated segments, the bending portion is typically covered with an elastic covering member. This covering member is made as flexible as possible to ensure that the motion of the bending portion is not impaired. An example of such a covering member is shown in EP 0 535 847.

Endoscopes are typically equipped with a vision system of some sort, traditionally based on a fiber optic cable between the tip of the endoscope and the control handle. Lately however, fiber optic cables have been replaced with electronic video cameras mounted directly at the tip of the endoscope, see for example US 2008/0091064. Video cameras are much cheaper and much more robust than fiber optic cables.

In certain cases, the endoscope could have one or more channels to enable passage of instruments (for example forceps or injection devices) or to enable the transport of a fluid to the tip of the endoscope.

Endoscopes are used for many different applications. One of these applications is to help in establishing artificial respiration for patients. An endotracheal tube is placed over the flexible insertion portion of the endoscope and the flexible insertion portion is then inserted into the airway of a patient. The vision system in the tip of the endoscope allows the endoscope to be guided into place without danger for the patient. Once the endoscope and the endotracheal tube are in place, the endoscope can be withdrawn leaving the endotracheal tube in place in the airway of the patient. In the current specification, the embodiments disclosed are directed to this application, but it should be understood that the teachings of the current specification can apply to many other types of endoscopes as well.

### SUMMARY OF THE INVENTION

It is a first aspect of the current invention to provide an endoscope which is better than the prior art endoscopes. In particular it is an aspect of the current invention to provide a bending portion which is of low cost.

This aspect is in part provided by an endoscope according to the introductory paragraph, wherein the elastic covering member is arranged such that it is stretched when the bending portion is in its neutral position. By stretched is meant that the elastic covering member is under tension when the bending portion is in its neutral position.

In one embodiment, the proximal end of the elastic covering member could be attached to the flexible insertion portion at a point, this attachment point being chosen such that the distance from the control handle to the attachment point is smaller than the distance from the control handle to the proximal end of the bending portion. In this way, the length of the elastic covering member can be made greater than L₂. By making the length of the elastic covering member greater than L2 in the neutral position of the bending portion, the percentage change in length of the elastic covering member when the bending portion is bent will be smaller than if the elastic covering member is made short. In this way, the forces due to the stretching of the elastic covering member can be reduced.

In one embodiment, the elastic covering member is arranged such that when the bending portion is bent the maximum amount, the portion of the elastic covering member arranged on the inside of the curvature of the bending portion is still under tension or at neutral. In this way, there will be no folding or bunching of the elastic covering member on the inside of the curve of the bending portion when the bending portion is bent.

The elastic covering member can be arranged displaceably with respect to the bending portion and/or the flexible insertion portion between the proximal end and the distal end of the elastic covering member. By displaceably is meant that the portion of the elastic covering member between the attachment point of the elastic covering member at the proximal end of the bending portion and the attachment point of the elastic covering member at the distal end of the bending portion, the elastic covering member is not connected to the bending portion and/or the flexible insertion portion such that the elastic covering member can freely displace and redistribute between the attachment points.

In order to reduce the outer diameter of the rigid tip portion, the diameter D1 of the flexible insertion portion at locations distal to the attachment point between the flexible insertion portion and the elastic covering member could be less than the diameter D2 of the flexible insertion portion proximal to said attachment point.

Another embodiment would be to choose the diameter D1 to be less than or equal to the diameter D2 minus two times the thickness of the elastic covering member.

The outer diameter D3 of the rigid tip portion could also be made greater than or equal to the outer diameter D4 of the bending portion plus two times the thickness of the elastic covering member. In this way the elastic covering member would be arranged flush with the outer surface of the rigid tip portion.

In one embodiment, the endoscope could further comprise a camera and/or a light source arranged in the rigid tip portion and the endoscope could further comprise a flexible printed circuit connected to the camera and/or the light source and in that the flexible printed circuit is arranged inside the bending portion of the endoscope such that the plane of the flexible printed circuit is perpendicular to the plane of the motion of the bending section. In this way, the resistance to motion of the flexible printed circuit to the motion of the bendable portion is reduced.

The flexible printed circuit could be electronically shielded by laminating the shielding material on either side of the flexible printed circuit.

In one preferred embodiment the bending portion could be composed of a plurality of plastic segments, each segment comprising at least two integrally moulded pins at one end and with at least two integrally moulded recess in the other end, said pins engaging with the recesses of an adjacent segment to allow the segments to pivot about the axis of the pins.

In order to allow the segments to snap together in a simple way, the pins could be formed with a surface which is arranged at an angle to the longitudinal axis of the segment between 20 and 70 degrees and in that said angle is arranged such that the distance between the longitudinal axis of the segment and the surface in the direction perpendicular to the longitudinal axis of the segment increases when moving along the longitudinal axis of the segment in the direction from the end comprising the pins to the end comprising the recesses. In the same manner, the segments could be formed with a surface between the recess and the edge of the segment closest to the recess, said surface being arranged at an angle to the longitudinal axis of the segment between 20 and 70 degrees and in that said angle is arranged such that the distance between the longitudinal axis of the segment and the surface in the direction perpendicular to the longitudinal axis of the segment increases when moving along the longitudinal axis of the segment in the direction from the end comprising the pins to the end comprising the recesses.

In one embodiment, the segments could be arranged as essentially cylindrically formed elements and the edges of the segments could be arranged with blunt edges in order to prevent cutting into the elastic covering member and/or an internal component of the endoscope in the case that the elastic covering member and/or an internal component of the endoscope comes between two connected segments.

In one embodiment, the segments could be made from a Liquid Crystal Polymer (LCP). This is advantageous to prevent the segments from being damaged during normal use.

It should be emphasized that the term “comprises/comprising/comprised of” when used in this specification is taken to specify the presence of stated features, integers, steps or components but does not preclude the presence or addition of one or more other features, integers, steps, components or groups thereof.

### BRIEF DESCRIPTION OF THE DRAWINGS

In the following, the invention will be described in greater detail with reference to embodiments shown by the enclosed figures. It should be emphasized that the embodiments shown are used for example purposes only and should not be used to limit the scope of the invention.

FIG. 1 shows a complete perspective view of an endoscope according to the current invention.

FIG. 2 shows a close-up perspective view of the bending portion of the endoscope of FIG. 1 in its neutral position and without the elastic covering member.

FIG. 3 shows a close-up perspective view of the bending portion of the endoscope of FIG. 1 in its most curved position and without the elastic covering member.

FIG. 4 shows a side view of the bendable portion of the endoscope of FIG. 1 in the neutral position.

FIG. 5 shows a side view of the bendable portion of the endoscope of FIG. 1 in its most curved position.

FIG. 6 shows a side view of a bendable portion of a second embodiment of an endoscope according to the invention in its neutral position.

FIG. 7 shows a side view of a bendable portion of a third embodiment of an endoscope according to the invention in its neutral position.

FIG. 8 shows a side view of a bendable portion of a fourth embodiment of an endoscope according to the invention in its neutral position.

FIG. 9 shows a close-up top perspective view of a single segment of the bending portion of the endoscope of FIG. 1.

FIG. 10 shows a close-up bottom perspective view of a single segment of the bending portion of the endoscope of FIG. 1.

FIG. 11 shows a cross section view of a single segment of the bending portion of the endoscope of FIG. 1 according to the line XI-XI defined in FIG. 12.

FIG. 12 shows a cross section view of a single segment of the bending portion of the endoscope of FIG. 1 according to the line XII-XII defined in FIG. 11.

### DETAILED DESCRIPTION OF THE EMBODIMENTS

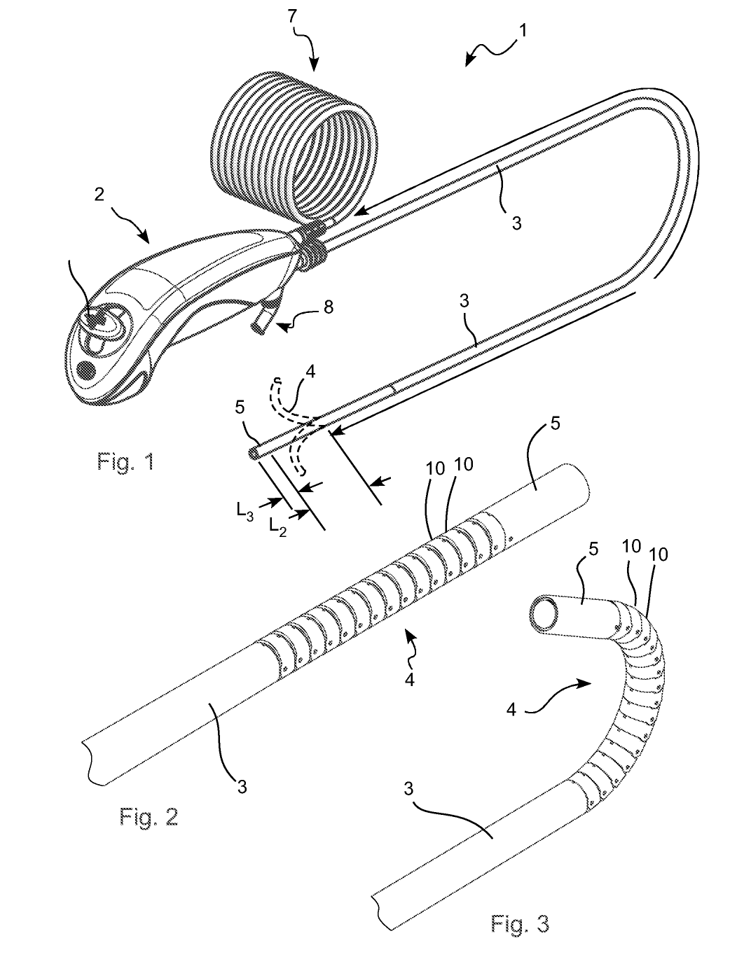

The endoscope 1 shown in FIG. 1 comprises a control handle 2, a flexible insertion portion 3 with length L₁, a bending portion 4 with length L₂ and a rigid tip portion 5 with length L₃. The control handle comprises a joystick 6 which is connected to the bending portion via control wires (not shown) arranged in the flexible insertion portion. When the joystick is activated, the bending portion bends as shown by the dashed lines in FIG. 1. The control handle further comprises an electrical cable 7 which is used to connect the endoscope to a power source and video monitor (not shown) and an injection port 8 which is in communication with an opening (not shown) at the tip of the endoscope. The injection port can be used to inject fluids out at the opening at the tip of the endoscope.

The flexible insertion portion 3 is arranged as a tube which is flexible and therefore bendable. However, the flexible insertion portion is stiff in the torsional and in the longitudinal direction. In this way, rotation of the control handle is transferred directly to the tip and allows the user of the endoscope to control the rotational position of the tip of the endoscope by twisting the control handle. A camera and a light source (not shown) are arranged in the rigid tip 5 of the endoscope. The electrical signals from the camera and the power to the camera and the light source are transferred via wires arranged in the flexible insertion portion and in the bending portion of the endoscope.

It should be noted that endoscopes comprising the above mentioned features are very well known to the person skilled in the art and further details will not be required by the person skilled in the art to understand and implement the current invention. The main invention disclosed in the current application is directed to the bending portion of the endoscope.

The bending portion shown in FIG. 1 is covered by an elastic covering member in the form of an elastic tube. The elastic covering member is made from an elastic material which allows the bending portion to bend without any great resistance. In FIGS. 2 and 3, the elastic covering member has been removed in order to show the mechanical details of the bending portion.

As can be seen from FIGS. 2 and 3, the bending portion 4 is comprised of a number of segments or links 10 linked together via corresponding pin and socket connections. This allows the successive segments to pivot with respect to each other. This type of articulated mechanism in bending portions of endoscopes is very common and many different types of bending portions are available.

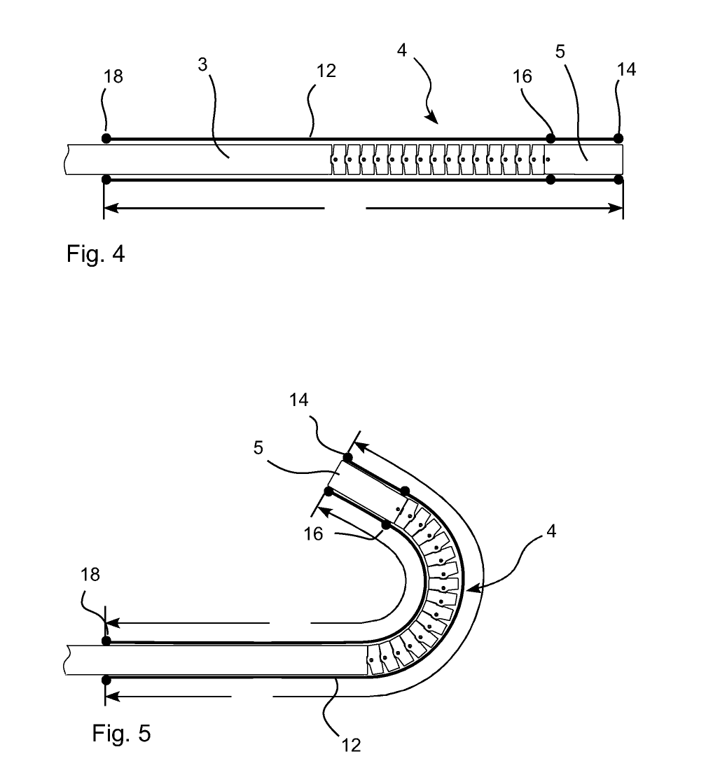

As mentioned previously, the bending section is covered by an elastic covering member. The elastic covering member is in the form of a tube much like a sleeve. The covering member has two main functions. Firstly it protects the bending portion from the environment in which it is present. For example it prevents bodily fluids from entering the interior of the endoscope. Secondly it protects the environment in which the bending portion is arranged from the bending portion itself by covering the uneven mechanical structure of the bending portion with a smooth cover. An elastic covering member 12 is shown in FIGS. 4 and 5.

One of the problems which is faced by the elastic covering member is that when the bending portion is bent, as shown in FIGS. 3 and 5, the length of the material arranged on the outside of the curvature is increased and the length of the material arranged on the inside of the curvature is decreased. This results in a stretching of the material on the outside of the curvature and a compression of the material on the inside of the curvature. The material which stretches resists the bending motion of the bending portion thereby requiring the user to apply more force to bend the section. On the other hand the material which compresses can fold and bunch up. This folding and bunching up causes an uneven surface on the endoscope making it more difficult to insert the endoscope. In addition, there is a risk that the folds will be caught between two segments of the bending portion. This could either result in the bending portion being blocked and/or the segments of the bending portion pinching the covering member and making a hole in the covering member.

The elastic covering member 12 of the current embodiment, as shown in FIGS. 4 and 5 is however pre-stretched such that even when the bending portion is bent the maximum amount, the material on the inner side of the curvature is still slightly under tension or just at neutral. In other words, the relaxed length of the elastic covering member before mounting is smaller than the length of the elastic covering member when mounted on the endoscope, even when bent a maximum amount. In this way, there is no bunching and/or folding. This can be better seen in FIGS. 4 and 5. When the bending portion is in its neutral position, the length of the covering member is L_{A}. When the bending portion is bent the maximum amount, the inner side of the curvature will have length L_{B} and the outer side will have length L_{C}. The length L_{C} is greater than the length L_{A} and the length L_{B} is less than the length L_{A}. Furthermore, if the elastic covering portion were measured in its relaxed state before mounting on the endoscope, its length would be equal to L_{B} or smaller.

It should be mentioned that a further advantage of this solution is that the elastic covering member can be used as a sort of return spring which seeks to put the bending portion into its neutral position when the control wires are relaxed.

Furthermore, as can be seen in FIGS. 4 and 5, the length of the elastic covering member is greater than the length of the bending portion. In this way, the change in length of the elastic covering member on the outside of the curvature is not so great when compared to the total length of the elastic covering member. In this way, the elastic covering member is not stretched any great amount when compared to its total length. The forces in the covering member are therefore not that great and the covering member does not restrict the motion of the bending portion as much as in the prior art.

In the current embodiment, the length of the bending portion is 4 cm and the length of the elastic covering member when mounted is 8 cm. In other words, the elastic covering member is twice the length of the bending portion. While a ratio of 2:1 between the length of the covering member and the length of the bending section is good, a greater or smaller ratio could also be imagined. However, the resistance to motion of the bending portion due to the elastic covering member will increase with decreased ratios.

It can also be noted from the figures that the elastic covering member in FIGS. 4 and 5 is attached to the rigid tip portion both at a point 14 at the most distal end of the tip portion and at a point 16 at the connection between the tip portion and the bending portion. The elastic covering member is then freely displaceable with respect to the rest of the bending portion and the flexible insertion portion until the attachment point 18 of the covering member and the flexible insertion portion. This allows the elastic covering member to freely slide on the bending portion and the flexible insertion portion in order to distribute itself in the best possible way depending on the current amount of curvature of the bending portion. In other words, it could be said that the unconnected length of the elastic covering member is greater than the length of the bending portion.

It should be noted that while it is advantageous to arrange the elastic covering member such that its length is greater than the length of the bending portion, it is not essential to the main teaching of the invention. The length of the elastic covering member could therefore be equal to the length of the bending portion, but be prestretched. If the elastic covering member is flexible enough, or if the forces to be transferred to the bending portion can be made large enough this solution would also work.

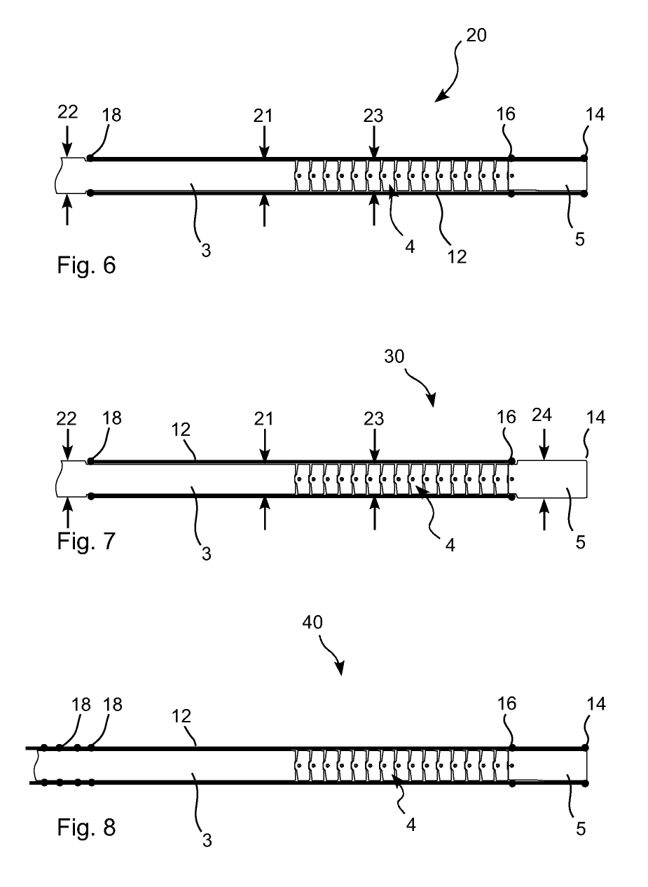

The embodiment of the endoscope 20 in FIG. 6 is for the most part identical to the embodiment shown in FIGS. 4 and 5 and therefore the same reference numerals are used for the same features. However in this case, the diameter 21 of the most distal portion of the flexible insertion portion 3 (ie the portion of the flexible insertion portion which is located distal to the attachment point between the flexible insertion portion and the elastic covering member) is slightly smaller than the diameter 22 of the remainder of the flexible insertion portion 3. The diameter 21 of the most distal portion of the flexible insertion portion is the same as the diameter 23 of the bending portion 4. The diameter 22 of the proximal portion of the flexible insertion portion is in this case equal to the diameter 23 of the bending portion plus two times the thickness of the elastic covering member 12. In this way, the elastic covering member 12 can be arranged such that its outer surface is flush with the outer surface of the most proximal portion of the flexible insertion portion. This avoids any unwanted edges on the shaft of the endoscope. An additional advantage of this embodiment is that the thinner diameter of the most distal portion of the flexible insertion portion is more flexible than the remainder of the flexible insertion portion. This allows the tip section of the endoscope to be more flexible and will result in more comfort to the patient.

The embodiment 30 of the endoscope of FIG. 7 is for the most part identical with the embodiment 20 of FIG. 6, however, in this case, the rigid tip portion 5 is also profiled in the same way as the flexible insertion element in order for the elastic covering member 12 to be arranged flush with the outer surface of the rigid tip 5. This embodiment allows the outer diameter 24 of the rigid tip portion to be made slightly smaller than in the case of the embodiment of FIG. 6 since the tip portion is not covered with the elastic covering member. This gives a more comfort to the patient.

In the embodiment 40 of an endoscope shown in FIG. 8, the elastic covering member 12 is arranged to cover the entire shaft of the endoscope including the rigid tip portion 5, the bending portion 4 and the entire flexible insertion portion 3. As shown in the figure, the elastic covering member is attached at its distal end to the rigid tip portion and the distal end of the bending portion. It is then attached at multiple points to the flexible insertion portion 3 at a point proximal to the proximal end of the bending portion. The multiple points ensure that the elastic covering member does not slide uncontrollably on the flexible insertion portion and bunch or fold undesirably.

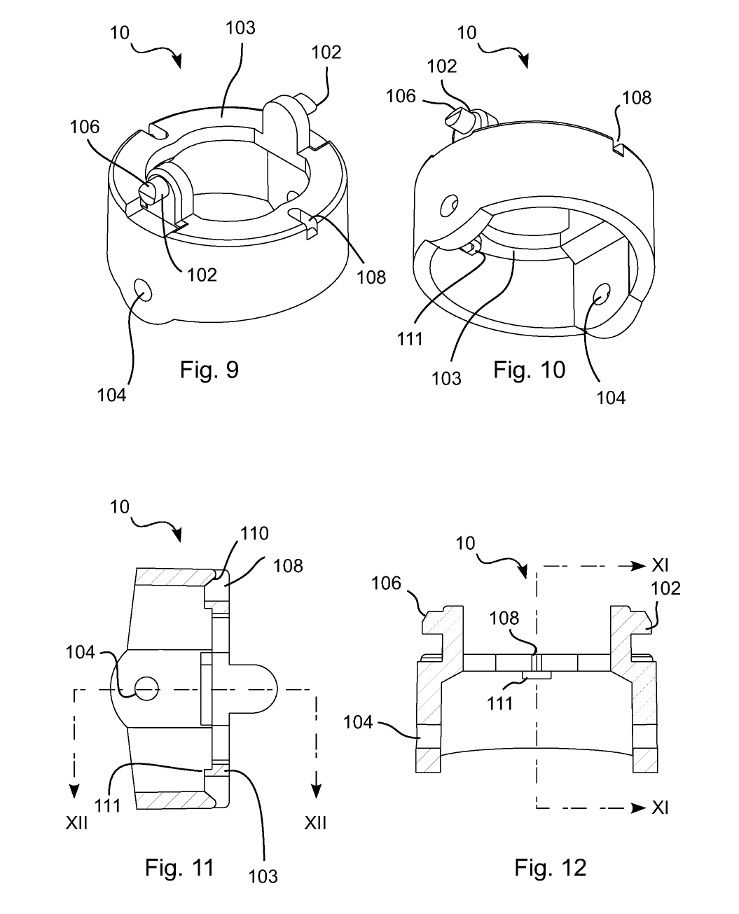

FIGS. 9-12 show detail views of the segments 100 used in the current embodiment of the bending portion. The segments are made via a plastic injection moulding process. In the current embodiment the segments are made from a liquid crystal polymer (LCP) due to the material properties of LCP. Each segment 100 comprises two pins 102 and two recesses or sockets 104. The pins are spaced at 180° to each other and the recesses are spaced at 180° to each other. In the current embodiment, the pins and recesses are arranged on a common plane and at each their end of the segment. However in the case where the bending section should bend in more than one plane, the pins could be rotated from the recesses by 90° . In this case, two sets of control wires could be used as is known in the prior art to provide a bending section which is movable in three dimensions.

As can be seen from the figures, the wall thickness of the segments is quite large when compared to the size of the segments. Furthermore, the edges of the segments are arranged to be blunt and not sharp. In this way, if the elastic covering member were to get trapped between two successive segments, the covering member would not be pinched and cut. Furthermore, if an inner object arranged inside the bending portion, for example a signal wire, where to be caught between two successive segments, no damage would occur to the inner object. The edge of the segment near the pin is furthermore arranged with a circular ledge 103 which is arranged around the entire periphery of the segment. This ledge increases the strength of the segment.

As can be seen from the figures, the pins 102 are provided with a tapered surface 106. The surface 106 is arranged such that when two successive segments are pressed together, the pins will automatically be displaced inwardly and thereby make it easier to connect the segments via a snap arrangement. If it is desired to separate the segments after joining together, the segments can be pressed together and forced into an elliptical shape, the pins can then be removed from the recesses. Instead of providing the pins with an angled surface, the recesses could be provided with an angled surface which would make it easier to click the links together.

It can also be seen from the figures, that each segment is provided with two through going channels 108 arranged 90° to the pins. These channels are provided to allow control wires to pass through the segments. The channels are tapered 110 at one end to provide a better passage for the control wire on the inner side of the curvature when the bending portion is most curved. In addition, the taper allows the control wires to be inserted into the channels in an easier manner. This makes assembly of the bending portion easier.

Another feature which can be seen from the figures is the extra material 111 around the channels 108 at the bottom of the circular ledge 103. The extra material is formed as a ridge along the outer periphery of the channel on the inner side of the segment. The purpose of this extra material is to provide a larger surface area for the contact between the segment and the control wire. It has been found that the control wires can cut into the plastic material of the segments if the thickness of the segments is too small at the point of contact. By increasing the amount of material at the control wire channel, the strength is improved but without a significant increase in weight or cost of the segment.

It should be noted that all the embodiments shown in the above description have shown segments with cylindrical cross sections. However, other forms of cross sections could also be imagined, for example square cross sections.

It should also be noted that the bending portion in the current invention has been made from injection moulded plastic links, however, the person skilled in the art will realize that the teaching of the main invention will also be applicable to other forms of links, for example metal links formed from plate element. In this respect it can be mentioned that the details of the plastic links disclosed in this specification, are different from the plastic links disclosed in the prior art. It could therefore be expected that the features of the plastic links which are different from the prior art could be subject of one or more divisional applications directed to this feature.

It is to be noted that the figures and the above description have shown the example embodiments in a simple and schematic manner. The internal electronic and mechanical details have not been shown in great detail since the person skilled in the art should be familiar with these details and they would just unnecessarily complicate this description.

1. An endoscope (1) comprising:
a. a control handle (2),
b. a flexible insertion portion (3) having length L₁ and being attached to the distal end of the control handle,
c. a bending portion (4) having length L₂ and being attached to the distal end of the flexible insertion portion,
d. a rigid tip portion (5) having length L₃ and being attached to the distal end of the bending portion,
e. a control mechanism arranged to control the bending of the bending portion, and
f. an elastic covering member (12) which is arranged to cover the bending portion and where the distal end of said elastic covering member is attached to the distal end (16) of said bending section and/or to said rigid tip portion (14) and where the proximal end of said elastic covering member is attached to the flexible insertion portion (18) and/or to the proximal end of the bending portion,
characterized in that the elastic covering member is arranged such that it is stretched when the bending portion is in its neutral position.

2. An endoscope (1) according to claim 1, characterized in that the proximal end of the elastic covering member (12) is attached to the flexible insertion portion (3) at a point (18), said attachment point being chosen such that the distance from the control handle (2) to the attachment point (18) is smaller than the distance from the control handle (2) to the proximal end of the bending portion (4).

3. An endoscope (1) according to claim 1 or 2, characterized in that the length of the elastic covering member (12) is greater than L₂.

4. An endoscope (1) according to claim 1, 2 or 3, characterized in that the elastic covering member (12) is arranged such that when the bending portion (4) is bent the maximum amount, the portion of the elastic covering member arranged on the inside of the curvature of the bending portion is still under tension or at neutral.

5. An endoscope (1) according to any one of claims 1-4, characterized in that the elastic covering member (12) is arranged displaceably with respect to the bending portion (4) and/or the flexible insertion portion (3) between the proximal end (18) and the distal end (16) of the elastic covering member.

6. An endoscope (1) according to claim 2, characterized in that the diameter D1 (21) of the flexible insertion portion (3) at locations distal to the attachment point (18) between the flexible insertion portion and the elastic covering member (12) is less than the diameter D2 (22) of the flexible insertion portion proximal to said attachment point.

7. An endoscope (1) according to claim 6, characterized in that the diameter D1 (21) is chosen to be less than or equal to the diameter D2 (22) minus two times the thickness of the elastic covering member (12).

8. An endoscope (1) according to any one of claims 1-7, characterized in that the outer diameter D3 of the rigid tip portion (5) is greater than or equal to the outer diameter D4 (23) of the bending portion (4) plus two times the thickness of the elastic covering member (12).

9. An endoscope (1) according to any one of claims 1-8, characterized in that the endoscope further comprises a camera and/or a light source arranged in the rigid tip portion (5) and in that said endoscope further comprises a flexible printed circuit connected to the camera and/or the light source and in that the flexible printed circuit is arranged inside the bending portion (4) of the endoscope such that the plane of the flexible printed circuit is perpendicular to the plane of the motion of the bending section.

10. An endoscope (1) according to claim 9, characterized in that the flexible printed circuit is electronically shielded by laminating shielding material on either side of the flexible printed circuit.

11. An endoscope (1) according to any one of claims 1-10, characterized in that the bending portion (4) is composed of a plurality of plastic segments (10), each segment comprising at least two integrally moulded pins (102) at one end and with at least two integrally moulded recesses (104) in the other end, said pins engaging with the recesses of an adjacent segment to allow the segments to pivot about the axis of the pins.

12. An endoscope (1) according to claim 11, characterized in that the pins (102) are formed with a surface (106) which is arranged at an angle to the longitudinal axis of the segment (10) between 20 and 70 degrees and in that said angle is arranged such that the distance between the longitudinal axis of the segment and the surface in the direction perpendicular to the longitudinal axis of the segment increases when moving along the longitudinal axis of the segment in the direction from the end comprising the pins to the end comprising the recesses.

13. An endoscope (1) according to claim 11 or 12, characterized in that the segments (10) are formed with a surface between the recess and the edge of the segment closest to the recess, said surface being arranged at an angle to the longitudinal axis of the segment between 20 and 70 degrees and in that said angle is arranged such that the distance between the longitudinal axis of the segment and the surface in the direction perpendicular to the longitudinal axis of the segment increases when moving along the longitudinal axis of the segment in the direction from the end comprising the pins to the end comprising the recesses.

14. An endoscope (1) according to any one of claims 11-13, characterized in that the segments (10) are essentially cylindrically formed elements and in that the edges (103) of the segments are arranged with blunt edges in order to prevent cutting into the elastic covering member (12) and/or an internal component of the endoscope in the case that the elastic covering member and/or an internal component of the endoscope comes between two connected segments.

15. An endoscope (1) according to any one of claims 11-14 characterized in that the segments (10) are made from a Liquid Crystal Polymer (LCP).
